(19) United States
(12) Patent Application Publication
(10) Pub. No.: US 2014/0039353 A1
(43) Pub. Date: Feb. 6, 2014

(54) Apparatus and Method of Analyzing Biomechanical Movement of an Animal/Human

(71) Applicant: Zig ZIEGLER, Phoenix, AZ (US)

(72) Inventor: Zig ZIEGLER, Phoenix, AZ (US)

(21) Appl. No.: 13/956,342

(22) Filed: Jul. 31, 2013

Related U.S. Application Data
(60) Provisional application No. 61/677,592, filed on Jul. 31, 2012.

(51) Int. Cl. A61B 5/11

(52) U.S. Cl. CPC A61B 5/11
USPC 600/595; 434/247

(57) ABSTRACT

A computer-executable method and an apparatus for analyzing biomechanical movement of an animal/human are used to detect and improve motion deficiencies being exhibited by the animal/human. The apparatus portion includes motion capture sensors, which are attached to a user's clothing or are directly adhered to the user's skin. The motion capture sensors are appropriately positioned across the user's body so that the computer-executable method is able to retrieve data for the user's full range of motion. From the data, the computer-executable method analyzes different physical movements, which include but not limited to sports skills and fitness exercises. The computer-executable method compares the data to an ideal version of a physical movement. The computer-executable method is also able to use the analysis of the data in order to create a performance report to illustrate the user's flaws while performing the physical movement and to suggest corrective drills.

The current application claims a priority to the U.S. Provisional Patent application Ser. No. 61/677,592 filed on Jul. 31, 2012.

### FIELD OF THE INVENTION

The present invention relates generally to a method and apparatus for capturing, measuring, and treating physiological deficiencies of human and animal motion. More specifically, the present invention is a method and apparatus that uses video and/or motion capture sensors or technology to accurately measure the biomechanics and kinesiology of human and animal motion or an individual as they perform physical movements including but not limited to sports skills, fitness exercises, running, and walking tasks and generates a computerized plan of treatment.

### BACKGROUND OF THE INVENTION

The present invention uses motion capture sensors or technology to accurately measure and improve muscular or joint strengths and weaknesses of the biomechanics and kinesiology of human and animal motion or an individual as they perform physical movements including but not limited to sports skills, fitness exercises, running, and walking tasks. From the data, the present invention performs the required biomechanical calculations and creates a detailed report with a computer generated list of exercises, treatments, or suggested activities to improve their ability to move efficiently and pain or injury free. The list may be in the form of text, pictures, or videos. If sensors are used, the present invention includes the placement of one or more biomechanics data measuring sensors, placed inside of an article of clothing or may be adhered to the skin.

The present invention's concept of use may be applied to golf, baseball, tennis, soccer, football, softball, running, walking, fitness exercises, physical therapy exercises and modalities, chiropractic adjustments and treatments, recommended medical injections and surgical procedures, yoga, acupuncture therapies, rehab exercises, and other yet to be discovered physical medicine related remedies, as well as the ability to turn any other human or animal motions or actions into a measurable biomechanical efficiency assessment with a grade range of 00.01% to 100%.

### BRIEF DESCRIPTION OF THE DRAWINGS

FIG. 1 is a simplified flow chart illustrating the general process from motion capture to analysis.

FIG. 2 is a simplified flow chart illustrating the automated monitoring process.

FIG. 3 is a diagram depicting some session drill variables being utilized by the present invention.

FIG. 4 is a block diagram depicting the apparatus and software components of the present invention.

FIG. 5 is a flow chart illustrating the general process for the present invention.

FIG. 6 is a continuation of the flow chart in FIG. 5.

FIG. 7 is a continuation of the flow chart in FIG. 6.

FIG. 8 is a flow chart illustrating a secondary process of how the apparatus components are used to capture motion data.

FIG. 9 is a flow chart illustrating a secondary process of how the software components are used to capture motion data.

FIG. 10 is a flow chart illustrating a secondary process of how raw trial data is collected and analyzed by the present invention.

FIG. 11 is a flow chart illustrating how recommendations are given based on deviation from the ideal data.

FIG. 12 is a flow chart illustrating a secondary process of how a performance report is compiled by the present invention.

FIG. 13 is a flow chart of the computerized physical-therapist process, which uses audible cues to make sure the corrective drills are done properly by the user.

FIG. 14 is a flow chart of the computerized physical-therapist process, which uses visual displays to make sure the corrective drills are done properly by the user.

### DETAILED DESCRIPTIONS OF THE INVENTION

All illustrations of the drawings are for the purpose of describing selected versions of the present invention and are not intended to limit the scope of the present invention.

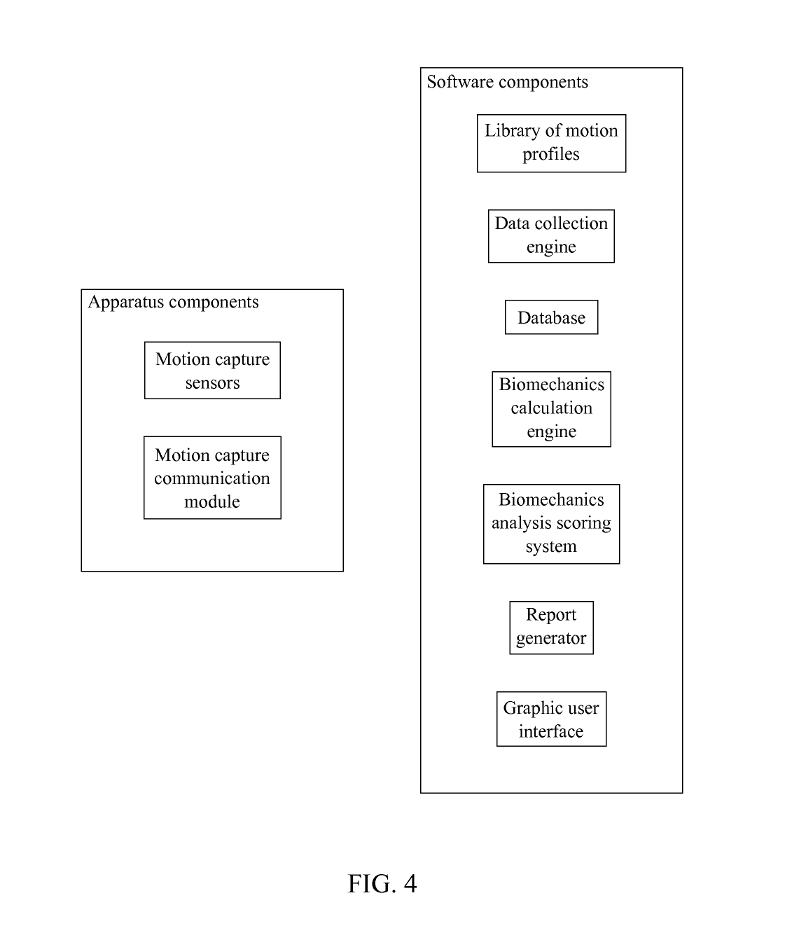

The present invention is a method of analyzing biomechanical movement of an animal/human in order to detect and improve motion deficiencies being exhibited by the animal/human. More specifically, the present invention is used to detect and improve motion deficiencies for a particular physical movement such as but not limited to sports skills, fitness exercises, running, and walking tasks. The method of the present invention is a software application, which is executed by computer-executable instructions stored on a non-transitory computer-readable medium. As can be seen in FIG. 4, the apparatus portion of the present invention includes a plurality of motion capture sensors, a motion capture communication module, and a computer that is capable of executing the software application. The apparatus portion of the present invention can be modified with more or less components in order to facilitate the method of the present invention. The plurality of motion capture sensors are positioned on or attached to specific limbs or joints of the animal/human so that a particular physical movement can be observed by the software application. The motion capture sensors can be placed inside an article of clothing or may be adhered to the skin. In general, the data from the motion capture sensors allows the present invention to perform the required biomechanical calculations. From this data, the present invention can create a computer generated list of corrective drills such as but not limited to exercises, treatments, or suggested activities, which improves the animal/human to efficiently perform the specific physical movement without pain or injury. The motion capture communication module handles all of the communication protocols between the plurality of the motion capture sensors and the computer. The motion capture communication module can be a video camera that is used to record the movement of the motion capture sensors or can be a wireless module that is used to receive data from the motion capture sensors. The computer is used to implement the software application, and the computer can be a desktop, a laptop, a smart-phone, a tablet personal computer, or any other computing device.

The two tables below describe the positioning for the motion capture sensors for particular physical movements. Level 1 and level 2 convey the complexity for the arrangement of motion capture sensors:

| LEVEL 1 | SENSOR PLACEMENT |
|---|---|
| 1 Sensor | |
| Standing squat - hands above head | Pelvis OR Torso |
| Pushup | Pelvis OR Torso |
| SL Balance - eyes open | Pelvis OR Torso. Testing thigh |
| Walking 3.0 mph | Pelvis OR Torso |
| Dyanamic flexibility test - standing hamstring | Thigh OR lower leg |
| Dyanamic flexibility test - supine hip ER/IR | Thigh |
| Dyanamic flexibility test - standing hip | Thigh |
| ABD/ADD | |
| Dyanamic flexibility test - SH ER/IR | Lower arm, Scapula |
| 2 Sensors | |
| Standing squat - hands above head | Pelvis & Torso, B Thighs, B lower |
| | legs, B feet, Pelvis & head, R thigh & |
| | R pelvis, etc |
| Pushup | Pelvis & Torso, Pelvis & Head, B |
| | elbows, B forearms |
| SL Balance - eyes open | Pelvis & Torso, Pelvis & Testing lower |
| | leg, Pelvis & testing thigh, Pelvis & |
| | testing foot |
| Walking 3.0 mph | Pelvis & Torso, B Thighs, B lower |
| | legs, B feet, Pelvis & head |
| Dyanamic flexibility test - Hamstring | Thigh & lower leg, Lower leg & pelvis |
| flexibility | |
| 3 Sensors | |
| Standing squat - hands above head | Pelvis/Torso/Head, B Thighs & Pelvis, |
| | B lower legs & Pelvis, B feet & pelvis |
| Pushup | Torso & B elbows, Pelvi/Torso/Head, |
| | Pelvis & elbows |
| SL Balance - eyes open | Pelvis/Torso/Head, Lower |
| | leg/Thigh/Pelvis |
| Walking 3.0 mph | Pelvis/Torso/Head, Thighs & pelvis, |
| | Lower legs & pelvis, |
| 4 Sensors | |
| Standing squat - hands above head | Pelvis/Torso/B Thighs, Pelvis/Torso/B |
| | lower legs, B Thighs/B Lower legs |
| Pushup | Pelvsi/Torso/B elbows |
| SL Balance - eyes open | Head/Pelvis/Torso/Testing Thigh, |
| | Pelvis/Testing Thigh/Lower leg/foot |
| Walking 3.0 mph | B Thighs/Pelvis/Torso, B Thighs/B |
| | feet, B Thighs/B lower leg |

| LEVEL 2 | SENSOR PLACEMENT |
|---|---|
| 2 Sensors | |
| SL Front-reach squat - hands on hips | Pelvis & Torso, Thigh & lower leg, |
| | Thigh & foot, Lower Leg & Foot, |
| | Testing thigh & pelvis |
| Pushup - one leg | Pelvis & Torso, Pelvis & Head, B |
| | elbows, B forearms |
| SL Balance - eyes closed OR Multi-directional | Pelvis & Torso, Pelvis & Testing lower |
| | leg, Pelvis & testing thigh, Pelvis & |
| | testing foot |
| Running @ 6.0 mph | Pelvis & Torso, B Thighs, B lower |
| | legs, B feet, Pelvis & head |
| 3 Sensors | |
| SL Front-reach squat - hands on hips | Pelvis/Torso/Head, Thigh/Lower |
| | leg/Pelvis, Thigh/lower leg/foot, |
| | Pelvis/Lower leg/foot |
| Pushup - one leg | Torso & B elbows, Pelvi/Torso/Head, |
| | Pelvis & elbows |
| SL Balance - eyes closed OR Multi-directional | Pelvis/Torso/Head, Lower |
| | leg/Thigh/Pelvis |
| Running @ 6.0 mph | Pelvis/Torso/Head, Thighs & pelvis, |
| | Lower legs & pelvis, Pelvis & feet |
| 4 Sensors | |
| SL Front-reach squat - hands on hips | Pelvis/Torso/Thigh/Lower leg, |
| | Pelvis/Torso/lower leg/Foot, |
| | Pelvis/thigh/lower leg/foot |
| Pushup - one leg | Pelvsi/Torso/B elbows |
| SL Balance - eyes closed OR Multi-directional | Head/Pelvis/Torso/Testing Thigh, |
| | Pelvis/Testing Thigh/Lower leg/foot |
| Running @ 6.0 mph | B Thighs/Pelvis/Torso, B Thighs/B |
| | feet, B Thighs/B lower leg |

In reference to FIG. 4, the software application is provided with system components in order to implement the method of the present invention. Those system components include a library of motion profiles, a data collection engine, a database, a biomechanics calculation engine, a biomechanics analysis scoring system, a report generator, and a graphic user interface. The library of motion profiles contains an ideal data set for each motion profile, which describes the ideal motion of body segments and joints during a particular physical movement. The data collection engine handles the flow of data collection from the motion capture sensors by communicating with the motion capture communication module. The data collection engine contains flags for determining flow control such as passive view or data collection. The database is a means for the software application to create a structure for storing the data from the motion capture sensors. The biomechanics calculation engine is used calculate the biomechanical measurables of the physical movement being performed by the user. The biomechanics analysis scoring system is used to compare the biomechanical measurables between the raw trial data and the ideal data. The report generator accumulates the information from the data, the actual motion profile, the analysis of that motion profile, and corrective drills into one comprehensive report. The graphic user interface allows the user and the software application to interact with each other.

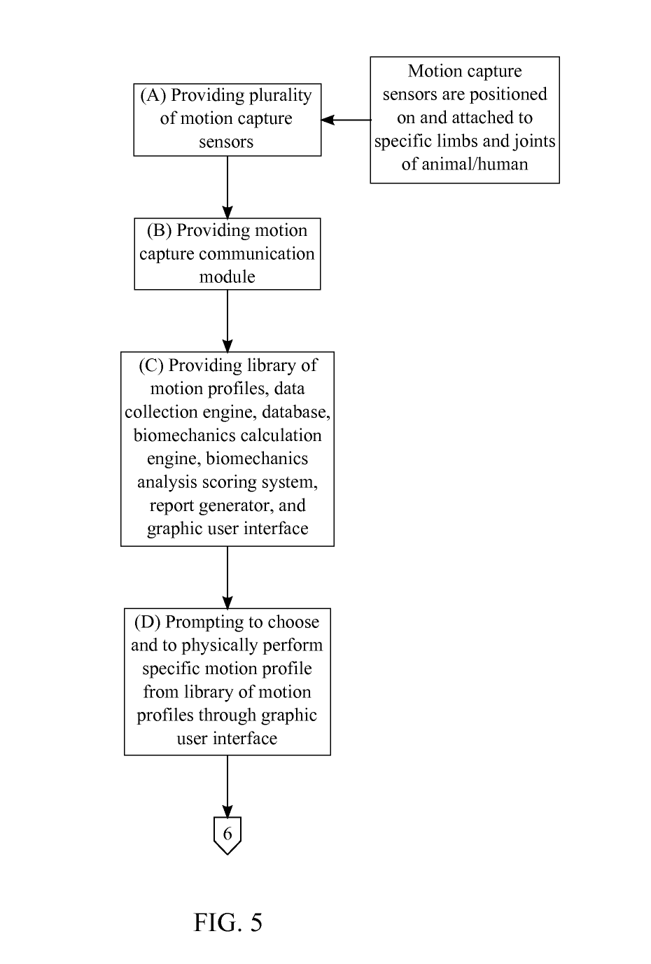

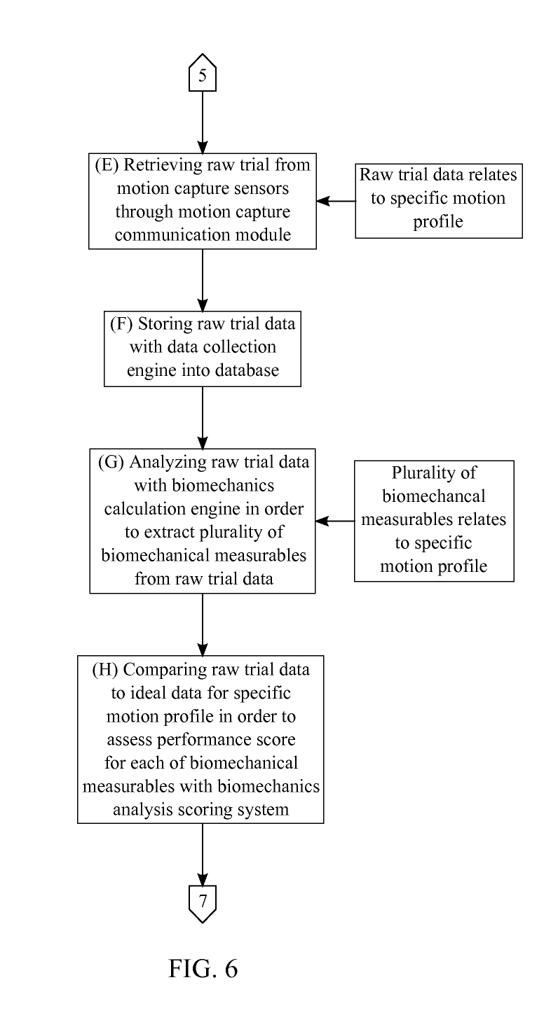

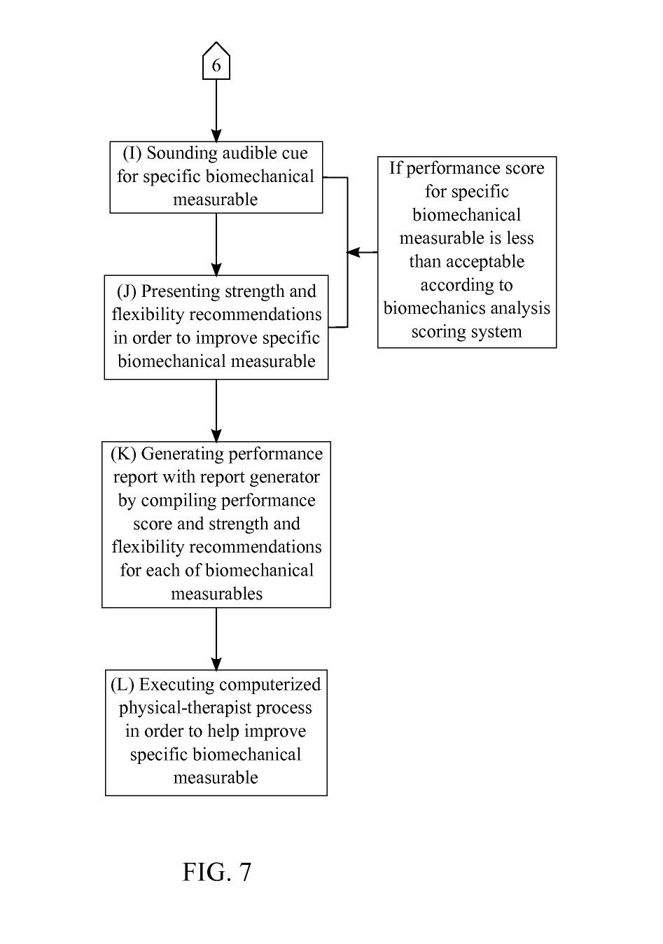

As can be seen FIGS. 5, 6, and 7, the software application follows a general process for the method portion of the present invention. The general process begins by prompting the user to choose a specific motion profile from the library of motion profiles through the graphic user interface. The software application will then prompt the user to physically perform the specific motion profile through the graphic user interface while the motion capture sensors are positioned on or attached to the user's limbs and joints. The general process continues by retrieving raw trial data from the motion capture sensors through the motion capture communication module. The raw trial data records the user's movement while the user is performing the specific motion profile. The software application will use the data collection engine to store the raw trial data within the database so that the raw trial data can be accessed at a later time.

The general process continues by analyzing the raw trial data with the biomechanics calculation engine in order to extract a plurality of biomechanical measurables that relates to the specific motion profile. The biomechanical measurables are aspects of the user's physical movement that can be quantified from the raw trial data. The biomechanical measurables is a calculated output that is gathered and processed from the sensor readings of the raw trial data. The software application will then compare the raw trial data to the ideal data for the specific motion profile in order to assess a performance score for each of the biomechanical measurables. The performance score is assessed by using the biomechanics analysis scoring system. The performance score will determine how different the biomechanical measurables are from the ideal data for the specific motion profile. If the performance score for a specific biomechanical measurable is less than acceptable according to said biomechanics analysis scoring system, then the software will sound an audible cue that the specific biomechanical measurable is performed wrong by the user and will present the user with strength and flexibility recommendations in order to improve the specific biomechanical measurable. The audible cue can be, but are not limited to, an automated voice or a warning bleep. The software application will also generate a performance report with the report generator by compiling the performance score and the strength and flexibility recommendations for each of the biomechanical measurables. The performance report will allow the user to view the biomechanical measurables for the specific motion profile as a whole, and, thus, allow the user to choose which biomechanical measurables need to be improved over others. In addition, the software application will execute a computerized physical-therapist process in order to help improve the specific biomechanical measurable by suggesting and monitoring correction drills that are done by the user.

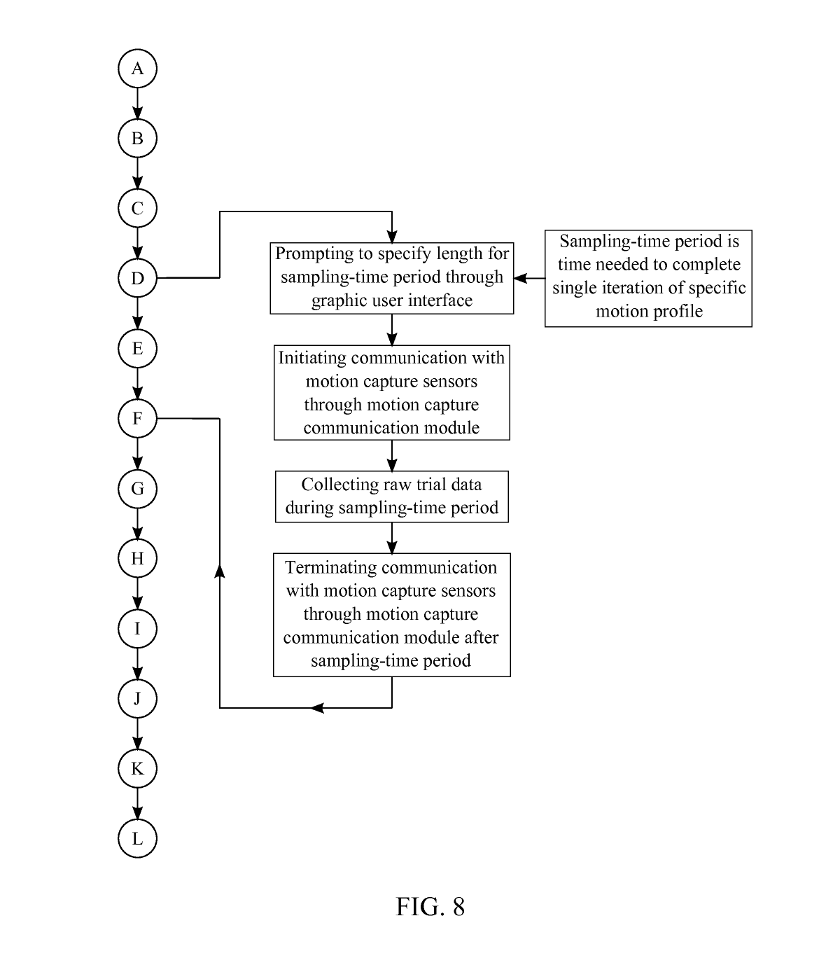

In reference to FIG. 8, the software application follows a secondary process while collecting the raw trial data from the motion capture sensors. This secondary process begins by prompting the user to specify a length for a sampling-time period through the graphic user interface, which is the length of time that is needed to complete a single iteration of the specific motion profile. In other embodiments, the length for the sampling-time period can be determined through a number of different means. The software application could prompt the user to start and end the collection of the raw trial data in order to retrieve the length for the sampling-time period. The software application could also directly retrieve the length of sampling-time period of as a part of the information that is provided with the specific motion profile. Once the length for the sampling-time period is known by the software application, the secondary process will continue by initiating communication with the motion capture sensors through the motion capture communication module. Consequently, the software application will begin collecting the raw trial data during the sampling-time period. After the duration of the sampling-time period, the software application will terminate communication with the motion capture sensors through the motion capture communication module, which will also mark the end of that trial.

The motion capture communication module is significantly used in the secondary process. The initialization of the motion capture communication module consists of any tasks that are necessary at startup time. The initialization of the motion capture communication module includes, but is not limited to, a one-time configuration of the necessary information, memory buffer allocation, and the private internal structures. When the initialization of the motion capture communication module is complete, the motion capture communication module will report success to the software application. The motion capture communication module will also allow the software application to access the library of motion profiles and other hardware manufacture-supplied libraries. The motion capture communication module will also report success to the software application when the communication is initiated with the motion capture sensors and when the communication is terminated with the motion capture sensors. In addition, if the motion capture communication module encounters an error while communicating with the motion capture sensors, then the motion capture communication module should report the error to the software application and shut down. An error string should report what the motion capture communication module was attempting to when the error occurred.

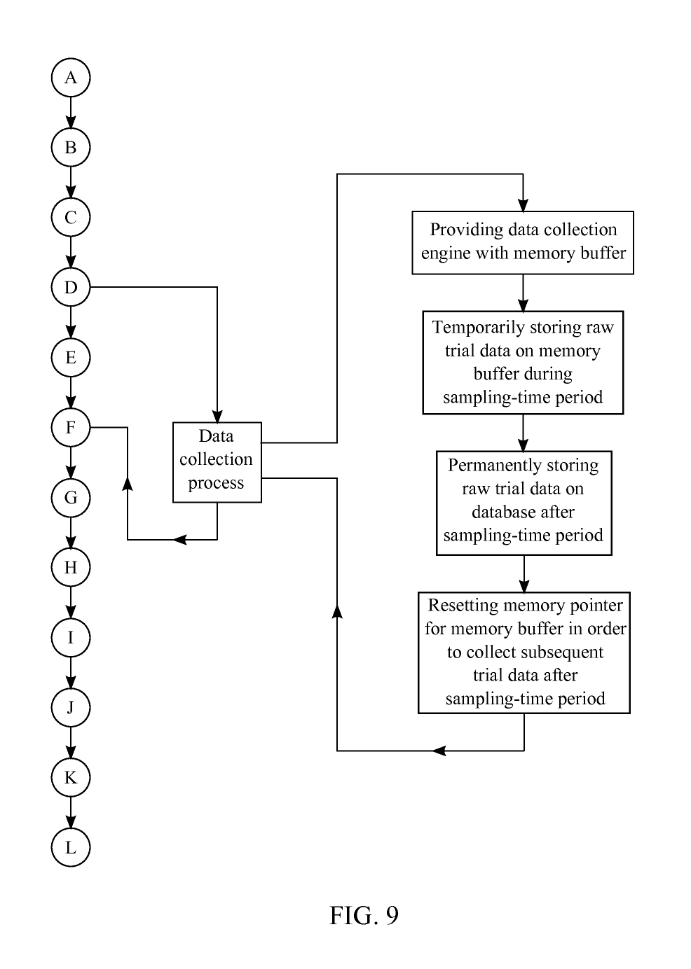

In reference to FIG. 9, the data collection process during each trial requires that the data collection engine only temporarily stores the raw trial data. Thus, the software application provides the data collection engine with a memory buffer, which resides within the data collection engine. In the preferred embodiment, the memory buffer should be treated as a ring buffer. The software application will temporarily store the raw trial data on the memory buffer during the sampling-time period. After the duration of the sampling-time period, the software application will then permanently store the raw trial data on the database. The software application will then reset the memory pointer of the memory buffer in order to collect subsequent trial data after the sampling-time period. Any data-overwrites for the memory buffer are the responsibility of the data collection engine to handle. The software application implements the data collection process by sending a start command, a stop command, and a reset command to the data collection engine.

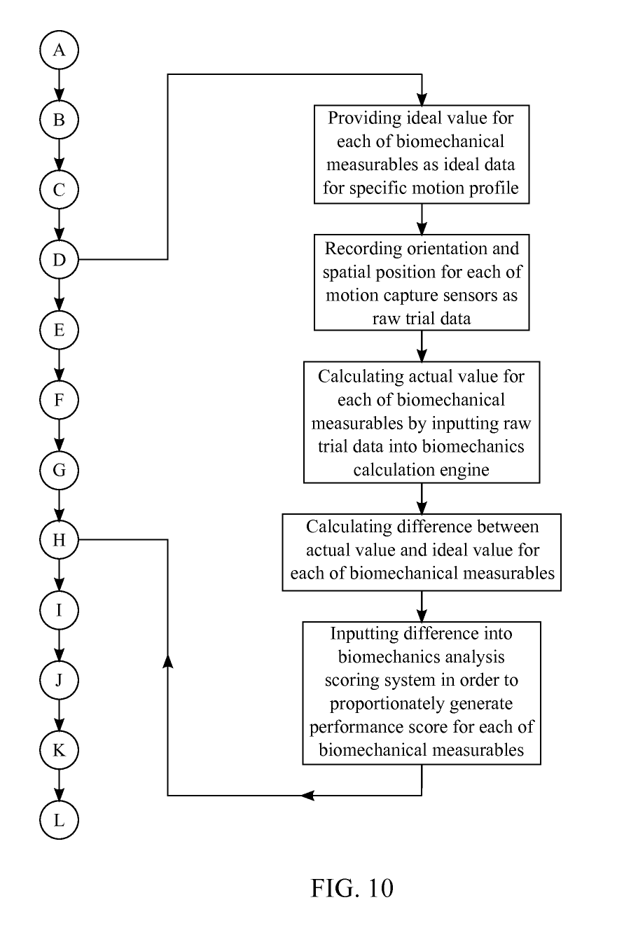

In order for the software application to calculate the biomechanical measurables, the software application needs to acquire certain kinds of information from the raw trial data, which is shown in FIG. 10. More specifically, the software application will record the orientation and spatial position of each motion capture sensor as the raw trial data while the user is performing the specific motion profile. The orientation and the spatial position of each motion capture sensor can be used to define three-dimensional Euler and Cardin angles. The software application will record the orientation and spatial position of each motion capture sensor during the entire length of the sampling time period. The software application will then calculate an actual value for the each of the biomechanical measurables by inputting the raw trial data into the biomechanics calculation engine. The actual value for each biomechanical measurable is the aspect of the user's physical movement that can be interpreted and measured by the software application. The ideal value for each biomechanical measurable is also provided to the software application because the ideal data for the specific motion profile contains the ideal value for each biomechanical measurable. The software application will then calculate a difference between the actual value and the ideal value for each of the biomechanical measurables, which allows a user to detect motion deficiencies in different biomechanical measurables. The difference for each biomechanical measurable is inputted into the biomechanics analysis scoring system in order to proportionately generate the performance score for each biomechanical measurable. Thus, if the difference for a specific biomechanical measurable is a large deviation between the actual value and the ideal value, then the performance score of that biomechanical measurable will be low. Similarly, if the difference for a specific biomechanical measurable is a small deviation between the actual value and the ideal value, then the performance score of that biomechanical measurable will be high.

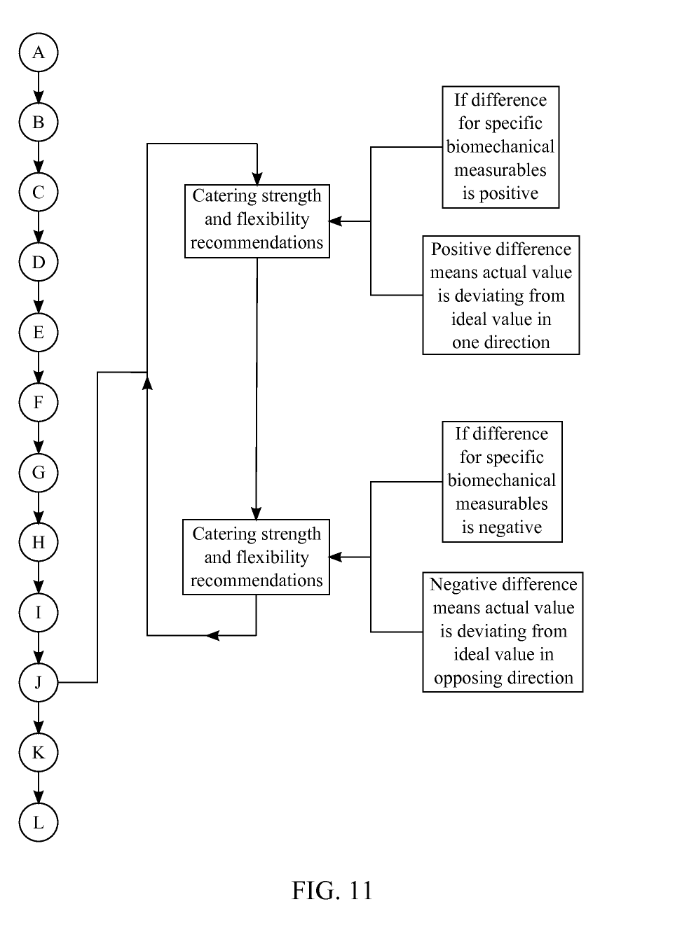

In reference to FIG. 11, the difference between the actual value and the ideal value allows the software to more accurately make strength and flexibility recommendations. If the difference for a specific biomechanical measurable is positive such that the actual value is deviating from the ideal value in one direction, then the software application will cater the strength and flexibility recommendations in order to minimize the difference between the actual value and the ideal value. Similarly, if the difference for a specific biomechanical measurable is negative such that the actual value is deviating from the ideal value in the opposite direction, then the software application will cater the strength and flexibility recommendations in order to minimize the difference between the actual value and the ideal value. For example, if the specific biomechanical measurable is the left lift angle for a user's leg and the difference between the actual value and the ideal value for the left lift angle is positive, the software application will recommend strengthening the user's abdominal muscles and loosening up the user's left hamstring. However, if the difference between the actual value and the ideal value for the left lift angle is negative, the software application will recommend strengthening the user's left hip flexor and loosening up the user's left glute and hamstring. The two tables below describe two biomechanical measurables for a running/walking example of the present invention:

### Left Lift Angle (Degrees)

| | | | | | | |
|---|---|---|---|---|---|---|
| | | | | | | Further |
| | | | | Strength | Flexibility | Assessment |
| | | | Deviation | recommen- | recommen- | Recommen- |
| Grade | Score | Degrees | from Norm | dations | dations | dations |

| | | | | | |
|---|---|---|---|---|---|
| Excellent | 100 Points | 38-44 | Normal | | |
| Good | 75 Points | 44.1-53 | Too much | Abdominal | Left hamstring |
| | | | | weakness | tightness |
| | | 29-37.9 | Too little | Left hip flexor | Left glute |
| | | | | weakness, right | tightness, |
| | | | | gastroc/soleus | left hamstring |
| | | | | weakness, right | tightness |
| | | | | quad weakness, | |
| | | | | right glute | |
| | | | | weakness, right | |
| | | | | hamstring weakness | |
| Fair | 40 Points | 53.1-65 | Too much | Abdominal | Left hamstring |
| | | | | weakness | tightness |
| | | 17-28.9 | Too little | Left hip flexor | Left glute |
| | | | | weakness, right | tightness, |
| | | | | gastroc/soleus | left hamstring |
| | | | | weakness, right | tightness |
| | | | | quad weakness, | |
| | | | | right glute | |
| | | | | weakness, right | |
| | | | | hamstring weakness | |
| Poor | 0 Points | 65.1-70 | Too much | Abdominal | Left hamstring |
| | | | | weakness | tightness |
| | | 0-19.9 | Too little | Left hip flexor | Left glute |
| | | | | weakness, right | tightness, |
| | | | | gastroc/soleus | left hamstring |
| | | | | weakness, right | tightness |
| | | | | quad weakness, | |
| | | | | right glute | |
| | | | | weakness, right | |
| | | | | hamstring weakness | |

### Right Lift Angle (Degrees)

| | | | | | | |
|---|---|---|---|---|---|---|
| | | | | | | Further |
| | | | | Strength | Flexibility | Assessment |
| | | | Deviation | recommen- | recommen- | Recommen- |
| Grade | Score | Degrees | from Norm | dations | dations | dations |

| | | | | | |
|---|---|---|---|---|---|
| Excellent | 100 Points | 38-44 | Normal | Abdominal | Right hamstring |
| | | | | weakness | tightness |
| Good | 75 Points | 44.1-53 | Too much | Right hip flexor | Right glute |
| | | | | weakness, left | tightness, |
| | | | | gastroc/soleus | left hamstring |
| | | | | weakness, left | tightness |
| | | | | quad weakness, | |
| | | | | left glute | |
| | | | | weakness, left | |
| | | | | hamstring weakness | |
| | | 29-37.9 | Too little | Abdominal | Right hamstring |
| | | | | weakness | tightness |
| Fair | 40 Points | 53.1-65 | Too much | Right hip flexor | Right glute |
| | | | | weakness, right | tightness, |
| | | | | gastroc/soleus | right hamstring |
| | | | | weakness, left | tightness |
| | | | | quad weakness, | |
| | | | | left glute | |
| | | | | weakness, left | |
| | | | | hamstring weakness | |
| | | 17-28.9 | Too little | Abdominal | Right hamstring |
| | | | | weakness | tightness |
| Poor | 0 Points | 65.1-70 | Too much | Right hip flexor | Right glute |
| | | | | weakness, right | tightness, |
| | | | | gastroc/soleus | right hamstring |
| | | | | weakness, left | tightness |
| | | | | quad weakness, | |
| | | | | left glute | |
| | | | | weakness, left | |
| | | | | hamstring weakness | |
| | | 0-19.9 | Too little | Left hip flexor | Left glute |
| | | | | weakness, right | tightness, |
| | | | | gastroc/soleus | left hamstring |
| | | | | weakness, right | tightness |
| | | | | quad weakness, | |
| | | | | right glute | |
| | | | | weakness, right | |
| | | | | hamstring weakness | |

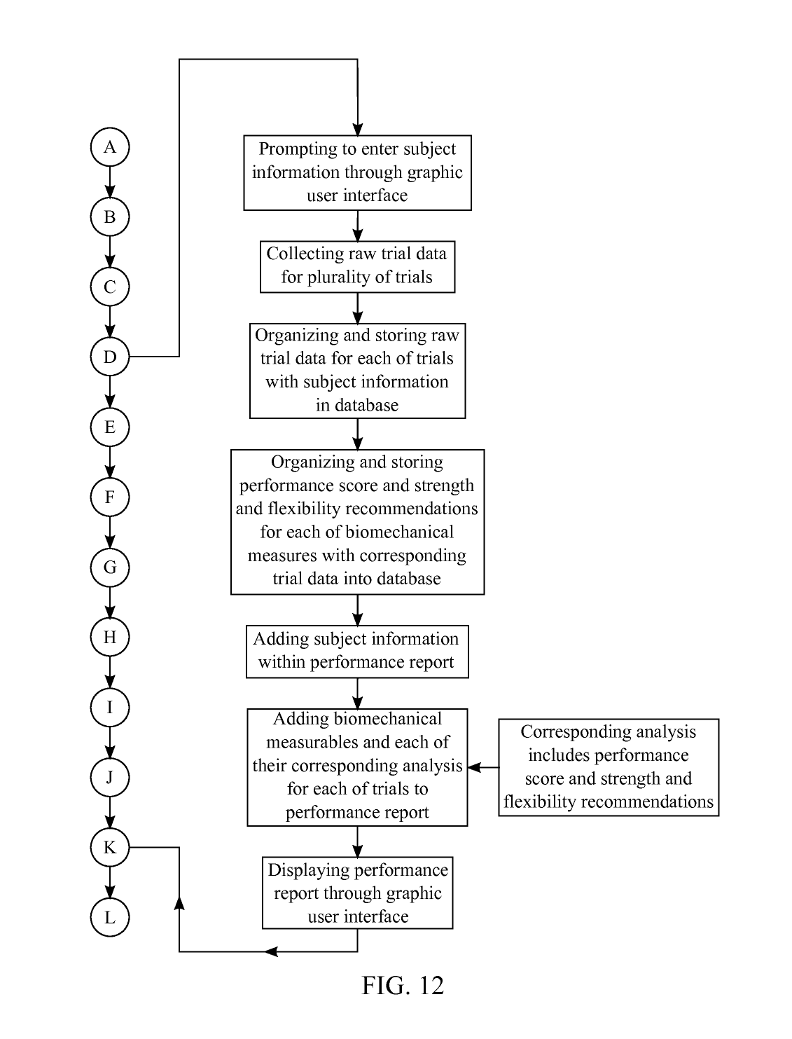

As can be seen in FIG. 12, the database allows the present invention to organize all of the information collected by the software application, which can collect raw trial data for a plurality of trials. In order to organize all of the information, the software application needs to prompt the user to enter the subject information through the graphic user interface. The subject information is any information that is particular to the user such as name, height, weight, and age. Once the software application receives the subject information, the software application organizes and stores the raw trial data for each trial with the subject information in the database. The software application also organizes and stores the performance score and the strength and flexibility recommendations for each biomechanical measurable with its corresponding raw trial data. The organization of the database allows the software application to easily compile the performance report with the report generator. First, the software application will add the subject information to the performance report, which allows anyone that reads the performance report with the report generator. Second, the software application will add the biomechanical measurables and each of their corresponding analysis for each of the trials to the performance report. The corresponding analysis includes the performance score and the strength and flexibility recommendations for each biomechanical measurable. After the performance report is completed by the report generator, the software application can then display the performance report to the user through the graphic user interface. The graphic user interface is capable of displaying all of the necessary graphical components for the raw trial data and the biomechanical measurables with their corresponding analysis for each trial. Those graphical components include but are not limited to data graphing, table generation, text boxes, and static bitmaps. In the preferred embodiment, a template file is used by the software application to create the performance report. In other embodiments of present invention, the report generator is able to compare the raw trial data and the biomechanical measurables with their corresponding analysis for each trial amongst the plurality of trials. The report generator could also be able to compare the raw trial data and the biomechanical measurables with their corresponding analysis for each trial to the data from other users.

In the preferred embodiment, the database is designed with a specific structure and organization. The database is to be created using an SQL based or other sufficient database program. All tables, forms, queries, code, and reports are created and/or controlled by the database. The database contains the following tables: subject information, raw trial data, and subject analysis data for each trial. The relationship of each table should be: one subject to many trials and one trial to one analysis, and one or multiple trial analysis to one or multiple trials analysis. The subject information table may contain any of the following fields: master key, subject identification, first name, middle name, last name, street address, city, state, zip code, phone number, email address, height, weight, date of birth, sport, coach's name, and coach's phone number, ability level, sport, sports implement dimensions, shoe sizes, injury history, dexterity, or other external variable which may assist the invention with generating an accurate report. The raw trial data table may contain the above and/or any of the following fields: subject identification, trial key, system, version, hardware, date of trial, time of trial, location, distance, conditions, sample rate, number of samples, number of sensors, and raw data sensor 1 through raw data sensor N. The only analysis currently supported would be the running analysis, golf swing analysis, pitching or throwing analysis, baseball/softball swing analysis, basketball shooting analysis, tennis groundstroke (forehand/backhand) analysis, tennis serve analysis, soccer kicking analysis, vertical leap or squatting analysis, and football throwing or kicking analysis.

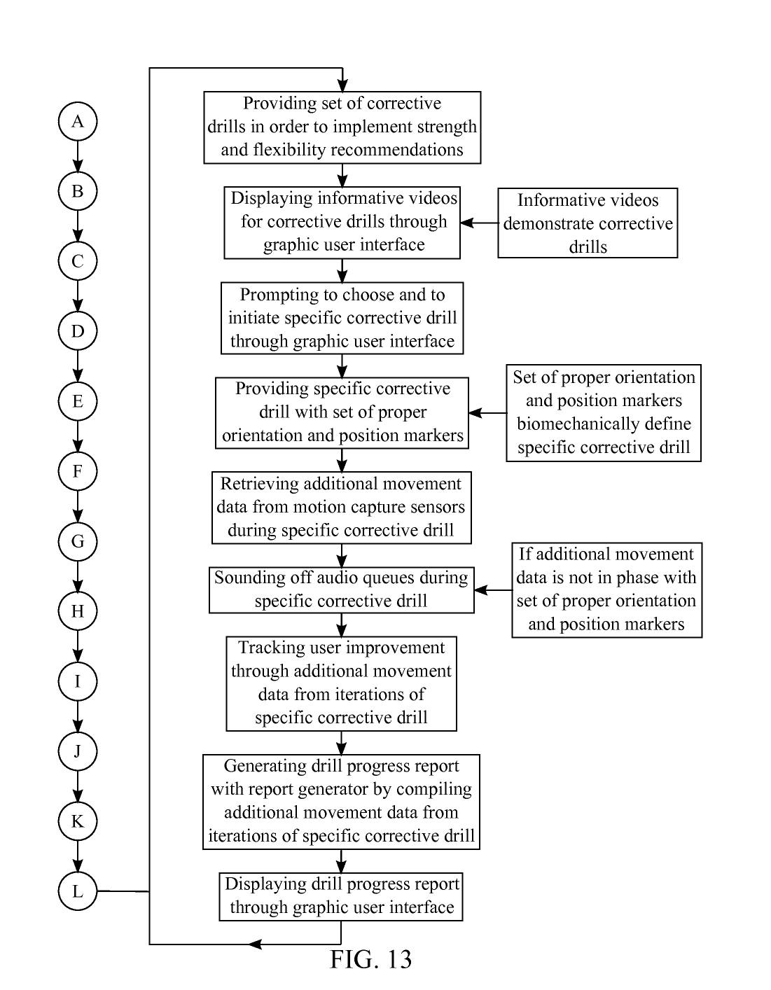

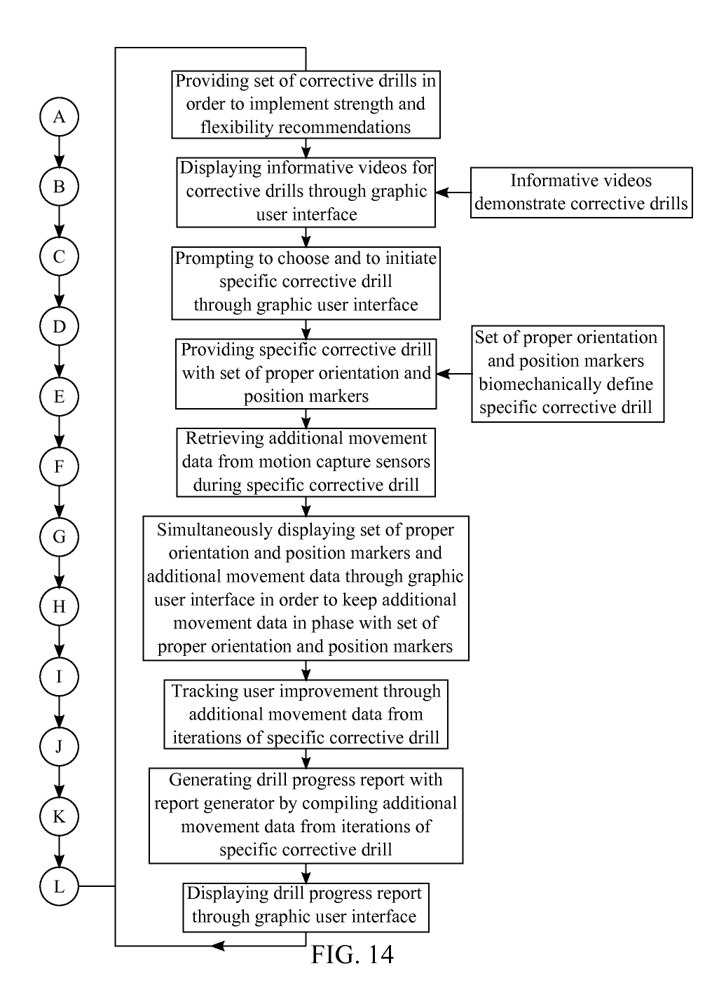

As can be seen in FIGS. 13 and 14, the computerized physical-therapist process is implemented as a means to improve the user's physical movement so that their biomechanical measurables are more similar to the ideal version of the physical movement shown in the specific motion profile. The process begins by suggesting a corresponding set of corrective drills in order to implement the strength and flexibility recommendations for each biomechanical measurable. The corrective drills are done by the user to improve on any weaknesses in their strength or flexibility, which would improve their physical movement while performing the specific motion profile. The process continues by displaying informational videos for the corrective drills to the user through the graphic user interface. The informational videos show the user how the corrective drills should be performed in order to improve their performance scores on particular biomechanical measurables and, thus, improve their physical movement. The process is also able to suggest a set of corresponding corrective treatments or procedures in order to implement the strength and flexibility recommendations for each biomechanical measurable. The corrective treatments or procedures include activities such as taking nutritional supplements or electric stimulation massages. The computerized physical-therapist process ends here if the user selects a treatment or procedure, but the process continues if the user selects to do a corrective drill. Thus, the software application will prompt the user to choose a specific corrective drill amongst all of the corrective drills that are provided, which is chosen by the user through the graphic user interface. The specific corrective drill is provided with a set of proper orientation and position markers, which defines how the user's body segments are supposed to be ideally oriented and ideally positioned while the user is performing the corrective drill. Once the user begins the specific corrective drill, the process will continue by retrieving additional movement data from the motion capture sensors while the user is performing the specific corrective drill.

The computerized physical-therapist process continues by implementing one of two methods in order to ensure the specific corrective drill is properly done by the user. One method is that the software application will sound off audio queues while the user is performing the specific corrective drill, which is show in FIG. 13. The software application will only sound the audio queues if the additional movement data is not in phase with the set of proper orientation and position markers. The audio queues are used to alert the user when the specific corrective drill is not being properly performed by the user's body segments. Consequently, sounding the audio queues will keep the additional movement data in phase with the specific motion profile. As can be seen in FIG. 14, another method is that the software application will simultaneously display both the set of proper orientation and position markers and the additional movement data on the graphic user interface, which will allow the user to view their physical movement in relation to the ideal physical movement of the specific corrective drill. This visual feedback from the graphic user interface allows the user to see when the specific corrective drill is not being properly done and allows the user to align their physical movement to the set of proper orientation and position markers. Consequently, the simultaneous display on the graphic user interface will also keep the additional kinesiological and biomechanical data in phase with the set of proper orientation and position markers. Both of these methods take advantage of the fact that every corrective drill can be broken down into specific phases and orientation markers. Finally, the software application will track the user improvement through the additional kinesiological and biomechanical movement data being collected during the iterations of the specific corrective drill. The software application can display the user improvement on a drill progress report, which is shown through the graphic user interface. The report generator is used to create the drill progress report by compiling the additional kinesiological and biomechanical movement data from the iterations of the specific corrective drill.

### Running/Walking Example:

One example of implementing the software application is for the running/walking case. The subject information and the performance analysis for running/walking should specifically comprise the following fields: subject identification, trial identification, analysis key, analysis type, total steps, total time, average step rate, time for each step 1 through n, total strides left, total strides right, average stride rate left, average stride rate right, times for left strides 1 through n, times for right strides 1 through n, average stride angle left, average stride angle right, stride angles for left 1 through n, stride angles for right 1 through n, max lift left, max lift right, average lift left, average lift right, left lift values 1 through n, right lift values 1 through n, max extension left, max extension right, average extension left, average extension right, left extension values 1 through n, right extension values 1 through n, left angular velocities 1 through n, right angular velocities 1 through n.

Additional analysis supported by the present invention includes body segment posture or position and orientation such as joint range of motion. The joint range of motion includes joint or bone flexion, extension, abduction, adduction, internal rotation, external rotation, pronation, supination, body segment, linear or angular velocity, body segment linear or rotational displacement, and GPS position data.

The data organization used by the software application facilitates the building of queries that generate performance report. The queries for report generation should allow a user to compare one of their biomechanical measurables to each of their other biomechanical measurables. The report generation should also allow the user to compare one of their biomechanical measurables to the data from other users and the other user's trials contained in the database. For example, compare the step rates of the current user to the step rates of all other users within a given age range.

For analyzing the biomechanical measurables, the first task is to find the minimum and maximum values along the curve. The system identifies each phase of the curve based on these values. It looks for the first minimum of each curve and then oscillates between positive and negative slopes.

In the running/walking case, the next task for analyzing the biomechanical measurables is to identify each step in order to determine a step rate. A step is defined as the maximum from the first curve to peak to the maximum of the second curve to peak. The next step is the maximum of the second curve to peak to the next maximum from the first curve to peak. This process repeats for the entire trial. This gives the system the total number of steps and the time between each step.

In the running/walking case, the next task for analyzing the biomechanical measurables is to identify each stride to get the stride rate. A stride is defined as the maximum of a curve to the next maximum of the same curve. This is done independently for each curve. This gives the system the number of strides for each curve and the time between each stride.

In the running/walking case, the next task for analyzing the biomechanical measurables is to compute the stride angle. The stride angle is defined as the difference between a minimum of the curve to the following maximum of the same curve. This is done independently for each curve.

In the running/walking case, the next task for analyzing the biomechanical measurables is to compute the maximum lift value for a curve. Lift values are defined as positive values on the curve. In the first task for analyzing the biomechanical measurables, the software application stores the value of each maximum for the curve. This function simply scans that list to find the highest value for the trial. This is done independently for each curve.

In the running/walking case, the next task of analyzing the biomechanical measurables is to compute the average lift value for a curve. Again, the data from the first step is used to compute this value. Average lift is the sum of all lift values divided by the number of values in the list. This is done independently for each curve.

In the running/walking case, the next task of analyzing the biomechanical measurables is to compute the maximum extension value for a curve. Extension values are defined as negative values on the curve. In the first task of analyzing the biomechanical measurables, the software application stores the value of each minimum for the curve. This function simply scans that list to find the most negative value for the trial. This is done independently for each curve.

In the running/walking case, the next task of analyzing the biomechanical measurables is to compute the average extension value for a curve. Again, the data from the first task is used to compute this value. Average extension is the sum of all extension values divided by the number of values in the list. This is done independently for each curve.

In the running/walking case, the next task of analyzing the biomechanical measurables is to compute the velocity for each curve. The first sample in velocity data is always zero. The next velocity value is computed by subtracting the value at T₁ from the value at T₀ and then dividing by the time difference between the samples. This is then the velocity value for the T₁ sample. This algorithm assumes uniform acceleration between each sample. This process is performed for the entire trial data.

In the running/walking case, the final task is to build the report text file. During this step the software computes the total time for the trial and generates the performance analysis with a biomechanics efficiency score, which is based on a comparison to the ideal biomechanics of the physical motion or sports skills

### Summarization of Invention:

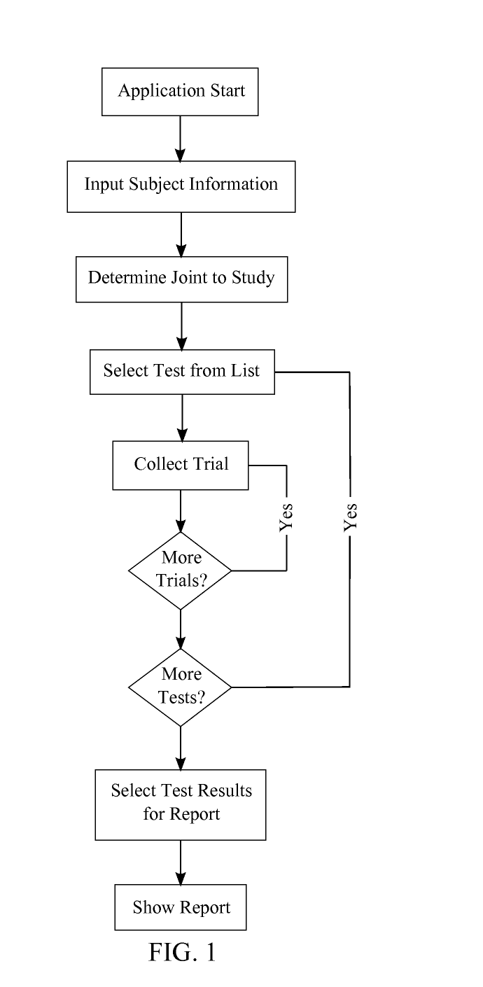

As seen in FIG. 1, the flow chart shown here illustrates a summarized version of the work flow for the software application. The idea for the design is to walk a user through the steps necessary to perform an analysis of a specific body joint or segment. At the start of the software application, the user should be presented with the options to open a data or subject file, input a new subject, or select a subject from the subject list. This is step one of the wizard. Once a subject is selected, the software application should present the user with a list of activities, exercises, joints or bone segments available for data collection and analysis. The list increases as support is added for more joints or actions. This is step two of the wizard. Next, the software application should present the user with a list of all available tests. The tests in the list increase as the user of the present invention adds support for more tests. This is the third and final step of the wizard. When the desired test is selected, the images depicting the data collected by the software application may appear on the graphic user interface, which is to be determined and created by the software application.

When the user finishes the data collection for a trial, the software application should ask the user to save the data and then ask if the user wishes to continue the data collection for more trials. If the user wants to perform more trials, then the software application returns to the data collection process. If the user does not want to perform more trials, then the software application asks the user if the user wishes to perform additional tests on the selected bone segment or joint. If the user agrees to perform additional tests, then the software application returns to the select test step. If the user does not want to perform additional tests, then the software application asks the user to select the test results from all tests performed to generate a performance report. If only one test is performed by the software application, then the software application should skip this step. From the desired selections in the above step, the software application generates a performance report for the trial(s). This performance report contains all information relevant to the tests perform. At this point, the user should have the option to close the program or return to the input subject step to continue collecting data for either the existing subject or a new subject. An overall score for accuracy and efficiency is given along with a breakdown of the accuracy within each user and/or trial.

The present invention performs the following operations to generate an automated report for any human or animal movement including the running/walking example:
1.) Collect motion data using motion capture sensors.
2.) Compute XY′Z″ sequence such that around x-axis represents flexion/extension and around y-axis represents abduction/adduction.
3.) Compute ZY′X″ sequence such that around z-axis represents internal/external rotation.
4.) Scan flexion/extension data for each bone or joint marking minimums and maximums. These values represent markers within the data file for computing steps and strides.
5.) Compute average maximums and minimums for each bone or joint.
6.) Compare each leg's values to the expected normative data.
7.) Based on the comparison above recommend a course of action including list of exercises that include the exercise in list format including number of sets, repetitions, and workload, resistance level, or duration that may correct any deficiencies in comparison to the expected range of motion or bone segment position/orientation identified by the system. The exercise list may include videos, performance description, or other components.

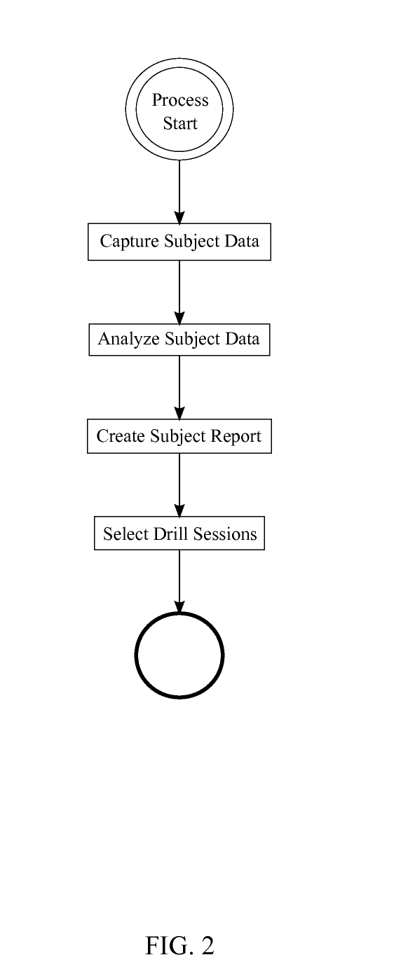

As can be seen in FIG. 2, the software application can record and analyze physical movements over longer periods of time such as an entire training session. For example, a user can have their baseball swing analyzed by the software application. From the performance report, the trainer or user gets a series of drills for use with the present invention. The trainer or the user can then configure the software application with the proper corrective drills. The software application automatically keeps track of how the user performs during those corrective drills. At the end of the training session, the software application reports how well the user performed the corrective drills both in accuracy and efficiency.

Traditionally, a therapist, a coach, or a trainer would give an instructive the lesson and rely on their eyes to determine if the user is accurately performing the corrective drill. However, the real time nature of the software application allows the trainer to assure the user that the user is performing the corrective drills in the proper manner.

The present invention has many applications in sports training and sports rehab. The software application can be used for physical movements in golf, basketball, baseball, tennis, soccer, football, softball, running, walking, fitness, and physical therapy and rehab exercises. The software application can basically be used to turn any human or animal motions or actions into a measurable biomechanical efficiency assessment.

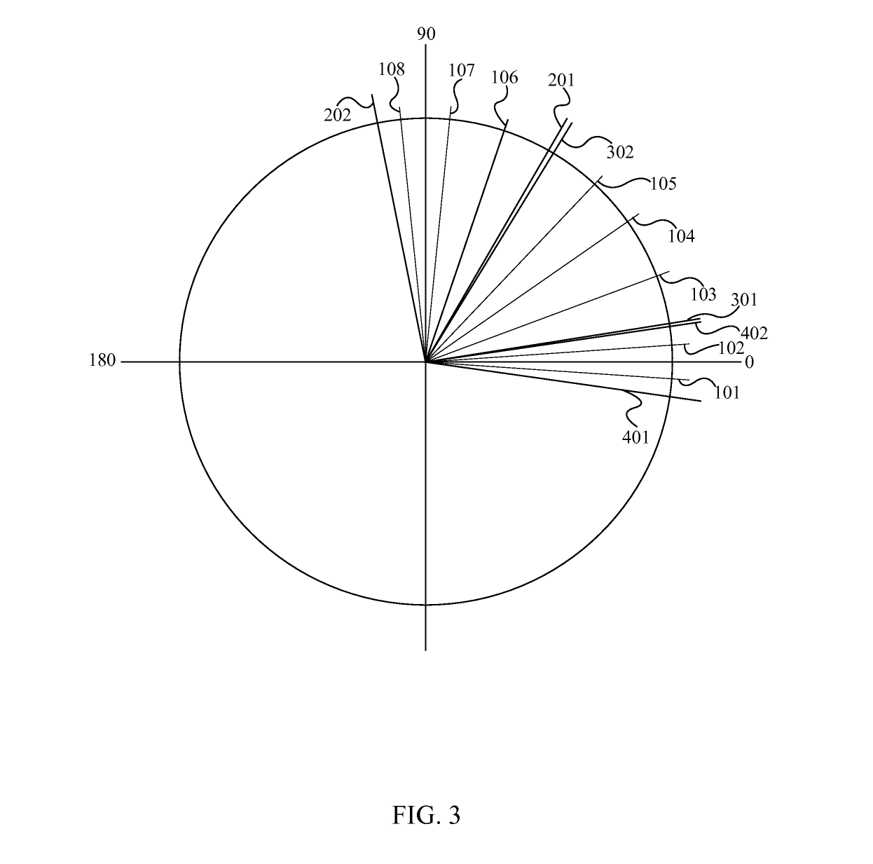

The software application can also manage a corrective drill with input from a physical trainer, a coach, or a kind of physical technician. As seen in FIG. 3, the diagram shows how a corrective drill can be defined to use with the software application. First, the technician must break up the desired corrective drill into a series of phases that can be defined using one calculation for orientation. In this example, the present invention uses rotation about the vertical axis of the body. The two lines 401, 402 represent the first phase of the corrective drill. The two lines 301, 302 represent the second phase of the corrective drill. The two lines 201, 202 represent the third phase in the corrective drill. The lines 101, 102, 103, 104, 105, 106, 107, 108 between each of the phase lines represent important markers that are used to score the user while the user is doing the corrective drill. The trainer can decide how many times the user is required to perform the corrective drill.

Once the plurality of motion capture sensors have been placed on the user and the user is appropriately aligned, the software application can then determine the orientation of the user. The software application now monitors the user in real time in order to determine which phase of the corrective drill that the user is currently doing. As the user performs the corrective drill, the software application compares their orientation with that defined by the markers within the current phase. If the user's body does not match those markers at a particular point, the software application sounds an audio tone. When the user reaches the end of the final phase, the software application resets the internal markers for the next trial.

During the corrective drill, the software application also keeps track of how often the subject is on target. This information is used to generate a report at the end of the session to give feedback on how well the user performed the corrective drill. An overall score for accuracy and efficiency is given along with a breakdown of the accuracy within each defined phase.

For example, the corrective drill shown above might represent a hitting drill. As the user moves their hips through the swing, their pelvis posture is analyzed by the software application. If user has poor rotational posture during a phase of the swing, the user will hear a tone or audible cue from the software application so that the user knows the corrective drill is being done wrong. The goal for the user is to perform the corrective drill without hearing a tone (negative feedback). This can also be performed using positive feedback such as a tone, audible cue, or visual cue when the goal is accomplished.

Although the invention has been explained in relation to its preferred embodiment, it is to be understood that many other possible modifications and variations can be made without departing from the spirit and scope of the invention as hereinafter claimed.

What is claimed:

1. A method of analyzing biomechanical movement of an animal/human in order to detect and improve motion deficiencies of the animal/human by executing computer-executable instructions stored on a non-transitory computer-readable medium, the method comprises the steps of:
providing a plurality of motion capture sensors, wherein said motion capture sensors are positioned on and attached to specific limbs and joints of an animal/human;
providing a motion capture communication module;
providing a library of motion profiles, a data collection engine, a database, a biomechanics calculation engine, a biomechanics analysis scoring system, a report generator, and a graphic user interface;
prompting to choose and to physically perform a specific motion profile from the library of motion profiles through said graphic user interface;
retrieving raw trial data from said motion capture sensors through said motion capture communication module, wherein said raw trial data relates to said specific motion profile;
storing said raw trial data with said data collection engine into said database;
analyzing said raw trial data with said biomechanics calculation engine in order to extract a plurality of biomechanical measurables from said raw trial data, wherein said plurality of biomechanical measurables relates to said specific motion profile;
comparing said raw trial data to ideal data for said specific motion profile in order to assess a performance score for each of said biomechanical measurables with said biomechanics analysis scoring system;
sounding an audible cue for a specific biomechanical measurable, and
presenting strength and flexibility recommendations in order to improve said specific biomechanical measureable,
if said performance score for said specific biomechanical measurable is less than acceptable according to said biomechanics analysis scoring system;
generating a performance report with said report generator by compiling said performance score and said strength and flexibility recommendations for each of said biomechanical measurables; and
executing a computerized physical-therapist process in order to help improve said specific biomechanical measurable.

2. The method of analyzing kinesiological and biomechanical movement of an animal/human in order to detect and improve motion deficiencies of the animal/human by executing computer-executable instructions stored on a non-transitory computer-readable medium, the method as claimed in claim 1 comprises the steps of:
prompting to specify a length for a sampling-time period through said graphic user interface, wherein said sampling-time period is the time needed to complete a single iteration of said specific motion profile;
initiating communication with said motion capture sensors through said motion capture communication module;
collecting said raw trial data during said sampling-time period; and
terminating communication with said motion capture sensors through said motion capture communication module after said sampling-time period.

3. The method of analyzing biomechanical movement of an animal/human in order to detect and improve motion deficiencies of the animal/human by executing computer-executable instructions stored on a non-transitory computer-readable medium, the method as claimed in claim 2 comprises the steps of:
providing said data collection engine with a memory buffer;
temporarily storing said raw trial data on said memory buffer during said sampling-time period;
permanently storing said raw trial data on said database after said sampling-time period; and
resetting memory pointer for said memory buffer in order to collect subsequent trial data after said sampling-time period.

4. The method of analyzing biomechanical movement of an animal/human in order to detect and improve motion deficiencies of the animal/human by executing computer-executable instructions stored on a non-transitory computer-readable medium, the method as claimed in claim 1 comprises the steps of:
providing an ideal value for each of said biomechanical measurables as said ideal data for said specific motion profile;
recording orientation and spatial position for each of said motion capture sensors as said raw trial data;
calculating an actual value for each of said biomechanical measurables by inputting said raw trial data into said biomechanics calculation engine;
calculating a difference between said actual value and said ideal value for each of said biomechanical measurables; and
inputting said difference into said biomechanics analysis scoring system in order to proportionately generate said performance score for each of said biomechanical measurables.

5. The method of analyzing biomechanical movement of an animal/human in order to detect and improve motion deficiencies of the animal/human by executing computer-executable instructions stored on a non-transitory computer-readable medium, the method as claimed in claim 4 comprises the steps of:
catering said strength and flexibility recommendations,
if said difference for said specific biomechanical measurable is positive,
wherein a positive difference means said actual value is deviating from said ideal value in one direction; and
catering said strength and flexibility recommendations,
if said difference for said specific biomechanical measurable is negative,
wherein a negative difference means said actual value is deviating from said ideal value in an opposing direction.

6. The method of analyzing biomechanical movement of an animal/human in order to detect and improve motion deficiencies of the animal/human by executing computer-executable instructions stored on a non-transitory computer-readable medium, the method as claimed in claim 1 comprises the steps of:
prompting to enter subject information through said graphic user interface;
collecting said raw trial data for a plurality of trials;
organizing and storing said raw trial data for each of said trials with said subject information in said database;
organizing and storing said performance score and said strength and flexibility recommendations for each of said biomechanical measures with corresponding trial data into said database;
adding said subject information within said performance report;
adding said biomechanical measurables and each of their corresponding analysis for each of said trials to said performance report, wherein said corresponding analysis includes said performance score and said strength and flexibility recommendations; and
displaying said performance report through said graphic user interface.

7. The method of analyzing biomechanical movement of an animal/human in order to detect and improve motion deficiencies of the animal/human by executing computer-executable instructions stored on a non-transitory computer-readable medium, the method as claimed in claim 1 comprises the steps of:
suggesting a corresponding set of corrective drills in order to implement said strength and flexibility recommendations;
displaying informative videos for said corrective drills through said graphic user interface, wherein said informative videos demonstrate said corrective drills;
prompting to choose and to initiate a specific corrective drill through said graphic user interface;
providing said specific corrective drill with a set of proper orientation and position markers, wherein said set of proper orientation and position markers biomechanically define said specific corrective drill;
retrieving additional movement data from said motion capture sensors during said specific corrective drill;
sounding off audio queues during said specific corrective drill,
if said additional movement data is not in phase with said set of proper orientation and position markers;
tracking user improvement through said additional movement data from iterations of said specific corrective drill;
generating a drill progress report with said report generator by compiling said additional movement data from said iterations of said specific corrective drill; and
displaying said drill progress report through said graphic user interface.

8. The method of analyzing biomechanical movement of an animal/human in order to detect and improve motion deficiencies of the animal/human by executing computer-executable instructions stored on a non-transitory computer-readable medium, the method as claimed in claim 1 comprises the steps of:
suggesting a corresponding set of corrective drills in order to implement said strength and flexibility recommendations;
displaying informative videos for said corrective drills through said graphic user interface, wherein said informative videos demonstrate said corrective drills;
prompting to choose and to initiate a specific corrective drill through said graphic user interface;
providing said specific corrective drill with a set of proper orientation and position markers, wherein said set of proper orientation and position markers biomechanically define said specific corrective drill;
retrieving additional movement data from said motion capture sensors during said specific corrective drill;
simultaneously displaying said set of proper orientation and position markers and said additional movement data through said graphic user interface in order to keep said additional movement data in phase with said set of proper orientation and position markers;
tracking user improvement through said additional movement data from iterations of said specific corrective drill;
generating a drill progress report with said report generator by compiling said additional movement data from said iterations of said specific corrective drill; and
displaying said drill progress report through said graphic user interface.

9. The method of analyzing biomechanical movement of an animal/human in order to detect and improve motion deficiencies of the animal/human by executing computer-executable instructions stored on a non-transitory computer-readable medium, the method as claimed in claim 1 comprises the steps of:
suggesting a corresponding set of corrective treatments or procedures in order to implement said strength and flexibility recommendations; and
displaying informative videos for said corrective treatments or procedures through said graphic user interface, wherein said informative videos demonstrate said corrective treatments or procedures.
